(19) United States
(12) Patent Application Publication
(10) Pub. No.: US 2012/0244208 A1
(43) Pub. Date: Sep. 27, 2012

(54) COSMETIC SKIN CARE METHODS AND COMPOSITIONS

(75) Inventor: Hugo Barrie Nel, Johannesburg (ZA)

(73) Assignee: Malan de Villiers, Wapadrand (ZA)

(21) Appl. No.: 13/394,066

(22) PCT Filed: Sep. 6, 2010
(86) PCT No.: PCT/IB2010/053999

§ 371 Date: May 25, 2012

Related U.S. Application Data
(60) Provisional application No. 61/240,048, filed on Sep. 4, 2009.

(51) Int. Cl. A61K 8/41; C07C 217/64; C07D 235/08; A61K 8/49; A61Q 19/08; C07C 251/58; A61K 8/40; A61K 9/127; A61K 9/51; A61K 38/40; C07C 217/70; C07D 203/10

(52) U.S. Cl. 424/450; 564/364; 514/653; 564/349; 514/652; 548/304.4; 514/394; 548/968; 514/183; 564/257; 514/640; 424/490; 514/18.8; 514/284

(57) ABSTRACT

This invention relates to a topical cosmetic skin care method and composition. The method and composition is for treating facial skin to cause contraction and tightening of the facial skin, by effecting contraction of the collagen of the skin using an active agent/s that induces fibroblast contraction of the extra cellular matrix of the skin. A preferred active agent is a beta-2-adrenergic receptor (beta2AR) inhibitor.

### BACKGROUND OF THE INVENTION

This invention relates to cosmetic skin care methods and compositions.

One of the well-recognized characteristics of ageing is sagging of the skin. This is due to a number of factors including loss of elasticity and firmness of the skin, the effect of gravity, the loss of skeletal support of the face, as well as loss of subcutaneous adipose tissue support in the face. These factors lead to jowl formation, sagging skin around the eyes, hollowing of the cheek-regions and wrinkles.

Objects of this invention are the provision of methods and compositions for the cosmetic treatment of sagging skin.

### SUMMARY OF THE INVENTION

A first aspect of this invention relates to a cosmetic method of treating skin, in particular treating facial skin to cause contraction and tightening of the facial skin, by effecting contraction of the collagen of the skin.

The skin may be treated by inducing fibroblast contraction of the extra cellular matrix of the skin to effect contraction of the collagen of the skin.

Active agents that cause collagen contraction may be selected from: cytokines and related biological mediators or parts thereof;

growth factors, for example Epidermal Growth Factor (EGF), Platelet Derived Growth Factor (PDGF) or Connective Tissue Growth Factor (CTFG);

prostanoids, for example Thromboxane A;

transferrins, for example Lactoferrin;

phospholipid derivatives, for example Lysophophatidic acid;

extra cellular matrix glycoproteins, for example Tenascin-C;

heat shock proteins (HSP), for example Heat Shock Protein 27; or part/s thereof.

According to a preferred embodiment of the invention, fibroblast contraction of the extra cellular matrix of the skin to effect contraction of the collagen of the skin may be induced by application, typically topical application, of a beta-2-adrenergic receptor (beta2AR) inhibitor.

Compounds having selective beta2 antagonist activity suitable for use in this invention include, but are not limited to, Butaxamine, erythro-dl-1-(7-methylindan-4-yloxy)-3-isopropylaminobutan-2-ol (ICI 118,551), H35/25, prenalterol, various 4- and 5-[2-hydroxy-3-(isopropylamino)propoxy]benzimidazoles, 1-(t-butyl-amino-3-ol-2-propyl)oximino-9 fluorene and various 2-(alpha-hydroxyarylmethyl)-3,3-dimethylaziridines.

Preferred selective beta-2-adrenergic receptor (beta2AR) inhibitors are the chemical erythro-dl-1-(7-methylindan-4-yloxy)-3-isopropylaminobutan-2-ol (ICI 118,551) and Butaxamine; most preferably erythro-dl-1-(7-methylindan-4-yloxy)-3-isopropylaminobutan-2-ol (ICI 118,551).

The active agent/s that cause collagen contraction are preferably administered topically.

The active agent/s that cause collagen contraction may be be palmitoylated, nanoencapsulated or liposomed, for example attached to a fatty acid such as palmitic acid, for example the peptide with the amino acid sequence YTRVVWXA attached to palmitic acid.

The treatment may comprise the use of at least two active agents in combination, for example a beta-2-adrenergic receptor (beta2AR) inhibitor and another active agent/s that causes collagen contraction, such as Lactoferrin or a part thereof.

The skin may be treated by inducing genetic changes associated with mechanical stress that cause collagen contraction.

Genetic changes associated with mechanical stress may be induced by an active agent which is the vectorized nucleotide sequence of the Shear Stress Response Element (SSRE) (i.e. GAGACC) or an activator of the SSRE promoter sequence.

A plant alkaloid, such as Glaucine, may be co-administered.

This invention also relates to, in a physiologically acceptable medium, a topical skin care composition, in particular treating facial skin to cause contraction and tightening of the facial skin, comprising an active agent capable of effecting contraction of the collagen of the skin.

The active agent is preferably capable of inducing fibroblast contraction of the extra cellular matrix of the skin to effect contraction of the collagen of the skin.

Active agents that cause collagen contraction may be selected from: cytokines and related biological mediators or parts thereof;

growth factors, for example Epidermal Growth Factor (EGF), Platelet Derived Growth Factor (PDGF) or Connective Tissue Growth Factor (CTFG);

prostanoids, for example Thromboxane A;

transferrins, for example Lactoferrin;

phospholipid derivatives, for example Lysophophatidic acid;

extra cellular matrix glycoproteins, for example Tenascin-C;

heat shock proteins (HSP), for example Heat Shock Protein 27; or a part thereof.

Preferably, the active agent is a beta-2-adrenergic receptor (beta2AR) inhibitor.

The beta-2-adrenergic receptor (beta2AR) inhibitor may be Butaxamine, erythro-dl-(7-methylindan-4-yloxy)-3-isopropylaminobutan-2-ol (ICI 118,551), H35/25, prenalterol, various 4- and 5-[2-hydroxy-3-(isopropylamino)propoxy]benzimidazoles, 1-(t-butyl-amino-3-ol-2-propyl)oximino-9 fluorene and various 2-(alpha-hydroxyarylmethyl)-3,3-dimethylaziridines.

Preferred selective beta-2-adrenergic receptor (beta2AR) inhibitors are the chemical erythro-dl-(7-methylindan-4-yloxy)-3-isopropylaminobutan-2-ol (ICI 118,551) and Butaxamine; most preferably erythro-dl-1-(7-methylindan-4-yloxy)-3-isopropylaminobutan-2-ol (ICI 118,551).

The active agent/s that causes collagen contraction may be palmitoylated, nanoencapsulated or liposomed, for example attached to a fatty acid such as palmitic acid, for example the peptide with the amino acid sequence YTRVVWXA attached to palmitic acid.

The composition may comprise at least two active agents in combination, for example a beta-2-adrenergic receptor (beta2AR) inhibitor and another active agent/s that causes collagen contraction, such as Lactoferrin or a part thereof.

The composition may contain an active agent/s capable of inducing genetic changes associated with mechanical stress that cause collagen contraction

The active agent may be a vectorized nucleotide sequence of the Shear Stress Response Element (SSRE) (i.e. GAGACC) or an activator of the SSRE promoter sequence.

The composition may contain a plant alkaloid, such as Glaucine.

The active agent/s may be present in an amount of 0.5 to 10%, preferably 0.5 to 5%, most preferably 1-2% by mass of the skin care composition.

Preferably, the skin care composition contains a plant alkaloid, such as Glaucine.

This invention further relates to the use of an active agent/s as described above capable of effecting contraction of the collagen of the skin in a method of manufacturing a cosmetic skin care composition for treating facial skin to cause contraction and tightening of the facial skin.

The composition may comprise at least two active agents in combination, for example a beta-2-adrenergic receptor (beta2AR) inhibitor and another active agent/s that causes collagen contraction, such as Lactoferrin or a part thereof.

Preferably, a plant alkaloid, such as Glaucine, is also used in the method of manufacturing the skin care composition.

This invention further relates to an active agent as described above capable of effecting contraction of the collagen of the skin for use in a method of treating skin, in particular for treating facial skin to cause contraction and tightening of the facial skin.

The invention also relates to a combination of at least two active agents as described above, for example a beta-2-adrenergic receptor (beta2AR) inhibitor and another active agent/s that causes collagen contraction, such as Lactoferrin or a part thereof.

### DESCRIPTION OF PREFERRED EMBODIMENTS

One of the well-recognized characteristics of ageing is sagging of the skin. This is due to a number of factors including loss of elasticity and firmness of the skin, the effect of gravity, the loss of skeletal support of the face, as well as loss of subcutaneous adipose tissue support in the face. These factors lead to jowl formation, saggy skin around the eyes, hollowing of the cheek-regions and wrinkles.

Due to this loss in firmness and tension in the skin, it can be understood that the mechanical stress in the skin also decreases. Mechanical stress on fibroblasts places them in a “synthetic” mode, by switching on genes for extra cellular matrix (ECM) production, as well as switching off genes for ECM breakdown. This should then lead to a vicious cycle of ECM loss, which further leads to loss in mechanical stress on fibroblasts. Aggravation of the characteristics of aged skin then follows.

According to the present invention, intervention in this process may be achieved by:
1) increasing the fibroblast contraction of the ECM, thereby causing collagen contraction in the skin increasing mechanical stress in the skin, making the skin firmer and younger; and/or
2) inducing genetic changes associated with mechanical stress.

Active agents that also cause collagen contraction in the skin may be:

cytokines or related biological matter;

growth factors, for example Epidermal Growth Factor (EGF), Platelet Derived Growth Factor (PDGF) or Connecitve Tissue Growth Factor (CTFG);

prostanoids, for example as Thromboxane A;

transferrins, for example Lactoferrin;

phospholipid derivatives, for example Lysophophatidic acid;

extra cellular matrix glycoproteins, for example Tenascin-C; and

heat shock proteins (HSP), for example Heat Shock Protein 27; or part/s thereof.

Fibroblast contraction of the ECM may be increased by beta-2-adrenergic receptor (beta2AR) inhibition. Fibroblast contraction of the extra cellular matrix of the skin to effect contraction of the collagen of the skin may be induced by application, typically topical application, of a beta-2-adrenergic receptor (beta2AR) inhibitor (also known as a selective beta2 antagonist). A beta-2-adrenergic receptor (beta2AR) inhibitor means an active agent having beta adrenergic blocking activity which is selective for beta2 -adrenergic receptors. Compounds having selective beta2 antagonist activity suitable for use in this invention include, but are not limited to, Butaxamine, erythro-dl-1-(7-methylindan-4-yloxy)-3-isopropylaminobutan-2-ol (ICI 118,551), H35/25, prenalterol, various 4- and 5-[2-hydroxy-3-(isopropylamino)propoxy]benzimidazoles, 1-(t-butyl-amino-3-ol-2-propyl)oximino-9 fluorene and various 2-(alpha-hydroxyarylmethyl)-3,3-dimethylaziridines. Preferred selective beta-2-adrenergic receptor (beta2AR) inhibitors are the chemical erythro-dl-1-(7-methylindan-4-yloxy)-3-isopropylaminobutan-2-ol (ICI 118,551) and Butaxamine.

Additional benefit could be gained by the fact that beta2AR inhibition also improves wound healing, improves skin barrier function and is anti-inflammatory.

A fragment of an active agent or related biological matter described above may be a peptide with the amino acid sequence YTRVVWXA (a fragment from Lactoferrin). This peptide at concentration of 1 microM has the effect of collagen gel contraction by fibroblasts, at about three times control. To facilitate skin penetration, said active may be palmitoylated or nano-encapsulated or liposomed. It is important to realize that fragments/parts of biological mediators may be biologically highly active. Therefore a peptide within the whole amino-acid sequence may be used to get a biological activity.

Genetic changes associated with mechanical stress may be induced by the Shear Stress Response Element (SSRE). The SSRE is encoded by the nucleotide sequence GAGACC. The active ingredient may be the vectorized nucleotide sequence of the SSRE or an activator of the SSRE promoter sequence.

Since it is known that collagen matrix contraction is associated with possible fibroblast apoptosis, an agent should be added in the formulation to counteract this effect. It is also known that beta2AR antagonism may cause lipogenesis. A state of decreased facial lipolysis may actually not be untoward in the context of the ageing face, but may not be an ideal side effect in all candidates. To this effect, the ideal combination with Glaucine may be considered, since it causes differentiation of pre-adipocytes into fibroblasts, thereby increasing the fibroblast population in the area and also has alpha-1 adrenergic antagonistic effects, which would counter-act lipogenesis.

In a preferred embodiment of the invention, ICI 118,551 (a beta2AR antagonist) is used in combination with YTRVVWXA (a peptide sequence found in Lactoferrin) attached to palmitic acid in a physiologically acceptable medium to prepare a skin care composition. Each active agent may be present in an amount of 0.5 to 10%, preferably 0.5 to 5%, most preferably 1-2% by mass of the skin care composition.

The clinical result of a cosmetic skin care product according to the invention is contraction and tightening of the facial skin. This is directly opposed to the effects of ageing on the skin. The result improves the appearance of wrinkles and excess facial skin, for which usually only a face-lift surgical procedure would be effective. Contraction of collagen is a beneficial effect sought by many other aesthetic non-surgical modalities, such as laser skin rejuvenation and thermal collagen contraction. A skin care product, whether a cream, gel, lotion of serum with skin-tightening effects, is a novel and simple solution. Such a product may also be formulated in combination with other active ingredients, to further enhance the anti-aging effect. Examples include Retinoids, anti-oxidants such as Vitamin C, Carnosine, Resveratrol, Niacinamide, Vitamin E, Alpha-lipoic acid, etc. Other examples include peptides, protease inhibitors, telomerase activators, molecular chaperones, anti-inflammatory agents, including NF-kappaB inhibitors. More examples are neuro-peptide modulators, moisturizers, including actives that increase Natural Moisturising Factor (NMF), Ceramides, Hyaluronic Acid and Aquaporins in the skin. Such a product may also have a beneficial effect on the appearance of cellulite. The active ingredients may be encapsulated or formed as nano-particles to enhance skin penetration. Skin penetration may also be enhanced by the use of an electromagnetic current or field.

The invention will now be described in more detail with reference to the following non-limiting examples:

### EXAMPLE 1

### Materials and Methods

### Primary Cell Culture

Normal human dermal fibroblasts (NHDFs) were obtained by tissue donation following circumcision following patient informed consent. The tissue was rinsed in Phosphate Buffered Saline (PBS) and Pen/Strep and Amphotericin B (Pen/strep) at 10 ug/ml. The tissue was then minced to 1 mm2 sections and suspended in Trypsin-EDTA 1:250 at room temperature (RT) for 1 hr. The tissue was then washed twice by centrifugation at 2500 g for 3min in serum supplemented DMEM/F12. A single cell suspension was created using mechanical disaggregation. The cells were counted using a haemocytometer and cultured at 1×106 cells per T75 tissue culture flask in DMEM/F12 containing 10% Fetal Bovine Serum (FBS) and 10 ug/ml Pen/strep. Incubators were kept at 37° C. and 5% CO2. Medium was changed every 3-4 days and cells were used from the 4th passage in vitro.

### Gel Formation

Collagen gels were formed. A solution of collagen, consisting of 2 mg/ml type 1 bovine collagen from bovine Achilles tendon in 0.5M acetic acid, was made. This made up 80% of the gel. Another 10% was made up of 10×PBS and the remaining volume was made up of FBS. The pH of the entire solution was adjusted to 7.4 using NaOH. 400 μl of the solution was placed into each well of a 24 well plate. The gels were set at 37° C. for 1 hr. After ensuring that the gels were set properly, they were detached from the sides of the wells and exposed to 1 ml fully supplemented DMEM/F12 overnight to acclimatize. The complete gels were seeded with 3.5×104 cells per well and the test compounds were added at this point. The test compound were added as follows: 0.1 mg/ml lactoferrin, 0.1 mg/ml ICI 118 551, 1.0 mg/ml Isoproterenol. A cell only and no cell control were also incuded.

### Measuring Gel Contraction

Photographs were taken from the same distance at 0, 3, 6, 12 and 24 hrs of exposure using a Canon Powershot A640 10MP camera. The photos were transferred to CorelDRAW X3 and the inside diameter of the well as well as the outer edge of the gel were measured using the circle tool. This was done for each well to ensure that the angle of incidence of the photo did not influence the result. These two diameters were then converted to a percentage of the well diameter by the formula geloutside/wellinside×100=gelrelative. This value was then worked out the same way for each gel at every time period. The time period Ohrs taken to be 100% of gel size and each time period after that was compared to it as a relative percentage. The standard deviation was calculated based on the original relative percentage to the well.

### Cytotoxicity

The cytotoxicity of isproterenol, ICI 118 551 and lactoferrin were determined using NHDFs as prepared above making use of the MTT (3-(4,5-Dimethylthiazol-2-yl)-2,5-diphenyltetrazolium bromide) assay in which a yellow terazole is converted to purple formazan by viable cells. 5×103cells/per well in a 96 well plate were seeded and allowed to attach overnight. They were then visually checked and exposed to varying concentrations of the test compounds for a 24 hr period. The results were read using a spectrophotometer at 570 nm. Viability was then calculated according to the standard methods set out in ISO 10993-1:2002.

### Results

### Contraction Study

Gel contraction was studied over a period of 24 hrs at intervals of Ohrs, 3 hrs, 6 hrs, 12 hrs and 24 hrs. All of the gels contracted over time. The control gels showed minimal contraction and started to return to their original sizes. The test gels, however, showed a steady decrease in diameter. The significant difference can be found in their rate of contraction while there is no significant difference in the final diameters of the gels. ICI 118 551 proved to contract the gels at a higher rate than its Lactoferrin and cells only controls.

A summary of the test results is shown in Table 1 below.

TABLE 1

| | 0 hrs | 3 hrs | 6 hrs | 12 hrs | 24 hrs |
|---|---|---|---|---|---|
| | % of original size (mean) | | | | |
| ISO (B-agonist) | 100 | 100.8622 | 79.6766 | 82.7588 | 83.79164 |
| LF (known collagen contractor) | 100 | 92.61855 | 64.47865 | 54.71446 | 49.73117 |
| ICI (B-blocker) | 100 | 66.04444 | 53.80149 | 51.33386 | 51.86021 |
| M (no drugs) | 100 | 81.73414 | 59.74771 | 49.83083 | 51.05158 |
| No cells or drugs | 100 | 92.24561 | 93.31738 | 91.21505 | 97.65967 |
| | STDev | | | | |
| ISO | 0.607155 | 2.597748 | 4.982024 | 2.304432 | 4.460034 |
| LF | 1.681224 | 4.193527 | 3.859579 | 4.413772 | 4.145883 |
| ICI | 1.033626 | 2.091728 | 4.871421 | 3.210674 | 2.961763 |
| M | 0.956106 | 3.513213 | 8.276265 | 6.763214 | 4.831194 |
| No cell | 3.341661 | 3.66116 | 5.009995 | 5.274445 | 3.602875 |

The dose response for ICI 118 551 was measured from 0.01 mg/ml to 0.2 mg/ml. Lower concentrations were found to not be significantly active while higher concentrations did not seem to increase the rate of contraction significantly. The ideal concentration was found to be 0.1 mg/ml on a gel containing 3.5×104 cells.

### Cytotoxicity

Cytotoxicity was measured using the MTT assay on NHDFs. Human dermal cells were used as it approximates the intended use. No cytotoxicity was found. The results are depicted in the graph below. Viability was monitored visually for 6 days at which point the cells exposed to Isoproterenol started to lose viability. All other cells maintained their viability over the six day period.

### Discussion

The rate of fibroblast seeded collagen type 1 gel contraction is influenced by the addition of the B-Blocker ICI 118 551. Gels did not return to their original dimensions in the ICI 118 551, lactoferrin and cell contracted groups when monitored for a further 3 days.

### Example 2

### Formulations

A)

| | | | % by | |
|---|---|---|---|---|
| Phase | Ingredient | INCI | mass | Function |
| I | Enulgade PL | Cetearyl Glucoside | 3 | O/W Cream |
| | 68/50 | (and) Cetearyl | | Base SE |
| | | Alcohol | | |
| | Cutina PES | Pentaerythrityl | 2 | Sensoral |
| | | Distearate | | Wax |
| | Cutina CP | Cetyl Palmitate | 0.5 | Consistency |
| | | | | Factor |
| | Monomuls 90-O | Glyceryl Oleate | 1 | Lipid Layer |
| | 18 | | | Enhancer |
| | Cetiol Sensoft | Propylheptyl | 2 | Emolient |
| | | Caprylate | | |
| | Cetiol CC | Dicaprylyl | 3 | Emolient |
| | | Carbonate | | |
| | Myritol 331 | Cocoglycerides | 5 | Emolient |
| | Cegesoft PFO | Passiflora Incarnata | 2 | Emolient |
| | | Seed Oil | | |
| | Covi-Ox T 70C | Tocopherol | 0.1 | Antioxidant |
| | Cosmedia SP | Sodium Polyacrylate | 0.7 | Sensorial |
| | | | | ploymer |
| II | Glycerin | Glycerin | 3 | Moisturizer |
| | Elestab 388 | | 1 | Preservative |
| | Eumulgin SG | Sodium Stearoyl | 0.5 | Co-Emulsifier |
| | | Glutamate | | |
| | Water | Water | balance | Diluent |
| III | ICI 118,551 | | 1 | Active |

B)

| | | | % by | |
|---|---|---|---|---|
| Phase | Ingredient | INCI | mass | Function |
| I | Enulgade PL | Cetearyl Glucoside | 3 | O/W Cream |
| | 68/50 | (and) Cetearyl | | Base SE |
| | | Alcohol | | |
| | Cutina PES | Pentaerythrityl | 2 | Sensoral |
| | | Distearate | | Wax |
| | Cutina CP | Cetyl Palmitate | 0.5 | Consistency |
| | | | | Factor |
| | Monomuls 90-O | Glyceryl Oleate | 1 | Lipid Layer |
| | 18 | | | Enhancer |
| | Cetiol Sensoft | Propylheptyl | 2 | Emolient |
| | | Caprylate | | |
| | Cetiol CC | Dicaprylyl | 3 | Emolient |
| | | Carbonate | | |
| | Myritol 331 | Cocoglycerides | 5 | Emolient |
| | Cegesoft PFO | Passiflora Incarnata | 2 | Emolient |
| | | Seed Oil | | |
| | Covi-Ox T 70C | Tocopherol | 0.1 | Antioxidant |
| | Cosmedia SP | Sodium Polyacrylate | 0.7 | Sensorial |
| | | | | ploymer |
| II | Glycerin | Glycerin | 3 | Moisturizer |
| | Elestab 388 | | 1 | Preservative |
| | Eumulgin SG | Sodium Stearoyl | 0.5 | Co-Emulsifier |
| | | Glutamate | | |
| | Water | Water | balance | Diluent |
| III | Palmitoyl- | | 1 | Active |
| | YTRVVWXA | | | |

C)

| | | | % by | |
|---|---|---|---|---|
| Phase | Ingredient | INCI | mass | Function |
| I | Enulgade PL | Cetearyl Glucoside | 3 | O/W Cream |
| | 68/50 | (and) Cetearyl | | Base SE |
| | | Alcohol | | |
| | Cutina PES | Pentaerythrityl | 2 | Sensoral |
| | | Distearate | | Wax |
| | Cutina CP | Cetyl Palmitate | 0.5 | Consistency |
| | | | | Factor |
| | Monomuls 90-O | Glyceryl Oleate | 1 | Lipid Layer |
| | 18 | | | Enhancer |
| | Cetiol Sensoft | Propylheptyl | 2 | Emolient |
| | | Caprylate | | |
| | Cetiol CC | Dicaprylyl | 3 | Emolient |
| | | Carbonate | | |
| | Myritol 331 | Cocoglycerides | 5 | Emolient |
| | Cegesoft PFO | Passiflora Incarnata | 2 | Emolient |
| | | Seed Oil | | |
| | Covi-Ox T 70C | Tocopherol | 0.1 | Antioxidant |
| | Cosmedia SP | Sodium Polyacrylate | 0.7 | Sensorial |
| | | | | ploymer |
| II | Glycerin | Glycerin | 3 | Moisturizer |
| | Elestab 388 | | 1 | Preservative |
| | Eumulgin SG | Sodium Stearoyl | 0.5 | Co-Emulsifier |
| | | Glutamate | | |
| | Water | Water | balance | Diluent |
| III | Palmitoyl- | | 1 | Active |
| | YTRVVWXA | | | |
| | ICI 118,551 | | 1 | Active |

1-62. (canceled)

63. A cosmetic method of treating skin, wherein fibroblast contraction of the extra cellular matrix of the skin to effect contraction of the collagen is induced by application of a beta-2-adrenergic receptor (beta2AR) inhibitor.

64. The method as claimed in claim 63, wherein the skin is facial skin and the method causes contraction and tightening of the facial skin.

65. The method as claimed in claim 63 or 64, wherein the beta-2-adrenergic receptor (beta2AR) inhibitor is selected from Butaxamine, erythro-dl-1-(7-methylindan-4-yloxy)-3-isopropylaminobutan-2-ol (ICI 118,551), H35/25, various 4- and 5-[2-hydroxy-3-(isopropylamino)propoxy]benzimidazoles, 1-(t-butyl-amino-3-ol-2-propyl)oximino-9 fluorene or a 2-(alpha-hydroxyarylmethyl)-3,3-dimethylaziridine.

66. The method as claimed in claim 65, wherein the selective beta-2-adrenergic receptor (beta2AR) inhibitor is the chemical erythro-dl-1-(7-methylindan-4-yloxy)-3-isopropylaminobutan-2-ol (ICI 118,551), or Butaxamine.

67. The method as claimed in claim 66, wherein the selective beta-2-adrenergic receptor (beta2AR) inhibitor is erythro-dl-1-(7-methylindan-4-yloxy)-3-isopropylaminobutan-2-ol (ICI 118,551).

68. The method as claimed in claim 63, wherein the beta-2-adrenergic receptor (beta2AR) inhibitor is administered topically.

69. The method as claimed in claim 63, wherein the beta-2-adrenergic receptor (beta2AR) inhibitor is palmitoylated, nanoencapsulated or liposomed.

70. The method as claimed in claim 69, wherein the beta-2-adrenergic receptor (beta2AR) inhibitor is attached to palmitic acid.

71. The method as claimed in claim 70, wherein a peptide with the amino acid sequence YTRVVWXA is attached to palmitic acid.

72. The method as claimed in claim 63, comprising the use of a beta-2-adrenergic receptor (beta2AR) inhibitor in combination with another active agent that causes collagen contraction.

73. The method as claimed in claim 72, wherein the other active agent that causes collagen contraction is Lactoferrin, or a part thereof.

74. The method as claimed in claim 63, wherein a plant alkaloid is co-administered.

75. The method as claimed in claim 74, wherein the plant alkaloid is Glaucine.

76. A topical cosmetic skin care composition in a physiologically acceptable medium, comprising a beta-2-adrenergic receptor (beta2AR) inhibitor capable of effecting contraction of the collagen of the skin.

77. The composition as claimed in claim 76, for treating facial skin to cause contraction and tightening of the facial skin.

78. The composition as claimed in claim 76, wherein the beta-2-adrenergic receptor (beta2AR) inhibitor is selected from Butaxamine, erythro-dl-1-(7-methylindan-4-yloxy)-3-isopropylaminobutan-2-ol (ICI 118,551), H35/25, various 4- and 5-[2-hydroxy-3-(isopropylamino)propoxy]benzimidazoles, 1-(t-butyl-amino-3-ol-2-propyl)oximino-9 fluorene or a 2-(alpha-hydroxyarylmethyl)-3,3-dimethylaziridine.

79. The composition as claimed in claim 78, wherein the selective beta-2-adrenergic receptor (beta2AR) inhibitor is the chemical erythro-dl-1-(7-methylindan-4-yloxy)-3-isopropylaminobutan-2-ol (ICI 118,551), or Butaxamine.

80. The composition as claimed in claim 79, wherein the selective beta-2-adrenergic receptor (beta2AR) inhibitor is erythro-dl-1-(7-methylindan-4-yloxy)-3-isopropylaminobutan-2-ol (ICI 118,551).

81. The composition as claimed in claim 76, wherein the active agent/s that cause collagen contraction is/are palmitoylated, nanoencapsulated or liposomed.

82. The composition as claimed in claim 81, wherein the beta-2-adrenergic receptor (beta2AR) inhibitor is attached to palmitic acid.

83. The method as claimed in claim 82, wherein a peptide with the amino acid sequence YTRVVWXA is attached to palmitic acid.

84. The composition as claimed in claim 76, comprising a beta-2-adrenergic receptor (beta2AR) inhibitor and another active agent/s that causes collagen contraction.

85. The composition as claimed in claim 84, wherein the other active agent/s that causes collagen contraction is Lactoferrin or a part thereof.

86. The composition as claimed in claim 76, containing a plant alkaloid.

87. The composition as claimed in claim 86, wherein the plant alkaloid is Glaucine

88. The composition as claimed in claim 76, wherein the/each active agent/s is present in an amount of 0.5 to 10% by mass of the skin care composition.

89. The composition as claimed in claim 88, wherein the/each active agent/s is present in an amount of 0.5 to 5% by mass of the skin care composition.

90. The composition as claimed in claim 87, wherein the/each active agent/s is present in an amount of 1 to 2% by mass of the skin care composition.

91. The use of a beta-2-adrenergic receptor (beta2AR) inhibitor capable of effecting contraction of the collagen of the skinin a method of manufacturing a cosmetic skin care composition for treating facial skin to cause contraction and tightening of the facial skin.
